(19) United States
(12) Patent Application Publication
(10) Pub. No.: US 2018/0364207 A1
(43) Pub. Date: Dec. 20, 2018

(54) NANOFIBER-BASED SENSORS AND APPARATUS FOR OXYGEN MEASUREMENT, METHOD OF MAKING AND METHOD OF USING THE SAME

(71) Applicant: ACCUFIBER TECHNOLOGIES LLC, Grove City, OH (US)

(72) Inventors: Collin S. ROBERTS, Springfield, OH (US); Ryan S. LANNUTTI, Grove City, OH (US); Greg A. MUNOZ, Columbus, OH (US)

(21) Appl. No.: 16/009,824

(22) Filed: Jun. 15, 2018

Related U.S. Application Data
(60) Provisional application No. 62/520,280, filed on Jun. 15, 2017.

(51) Int. Cl. G01N 31/22; G01N 33/00; G01J 3/02

(52) U.S. Cl. CPC G01N 31/225; G01J 3/0229; G01J 3/0218; G01N 33/0075

(57) ABSTRACT

A device or apparatus includes a light source for providing light, a sensing element, and a detector. The sensing element includes nanofiber, which includes at least one oxygen sensitive compound, and can be excited by the light from the light source to emit radiation. The detector detects the radiation emitted from the nanofiber, and then provides a measured concentration of oxygen in the sample. The device or apparatus may include an enclosure defining an opening for introducing a sample for testing. The sensing element is disposed inside the enclosure. The device or apparatus may optionally include a heating element including an optically transparent and electrically conductive material. The device or apparatus may also optionally include at least one optical filter, a porous mask, a pre-amplifier, and other components. The method of making the device or apparatus, and the method using the device or apparatus are also provided.

### PRIORITY CLAIM AND CROSS-REFERENCE

This application claims the benefit of U.S. Provisional Application No. 62/520,280, filed Jun. 15, 2017, which application is expressly incorporated by reference herein in its entirety.

### FIELD OF THE INVENTION

The disclosure relates to a sensor and an apparatus for quantitative measurement of oxygen. More particularly, the disclosed subject matter relates to a sensor and an apparatus comprising fibers, a method of making the same, and a method of using the same in wellness, health care, industry, defense, athletics and other applications.

### BACKGROUND

As a society, we are beginning to experience the advantages of real-time quantification of biological outputs from an individual to create a clearer picture of their well-being, health, and performance. Development of affordable, selective, and highly sensitive sensors enables widespread deployment of the corresponding small, portable, and cheap devices in diverse applications.

As an example, devices providing rapid breath analysis are desirable for applications in medical diagnosis, monitoring of caloric expenditure, athletic performance, therapy monitoring, military and defense applications or personalized lifestyle assessment. Existing techniques to monitor respiratory gases include mass spectroscopy, Raman spectroscopy, infrared light spectroscopy, infrared photoacoustics, piezoelectric resonance, polarography, fuel cell operation, paramagnetic analysis, and magnetoacoustics. Infrared systems are the most common.

The major disadvantages of conventional gas monitors include that they are typically large, fragile, and expensive; and require frequent calibration and maintenance. Managing the power consumption of these forms of atmospheric sensing is also a major issue. The mass of existing sensor systems is similarly problematic as each can include several pounds of hardware and sophisticated electronics. Added to this is that the footprint of each of these devices can be relatively large. Finally, the speed with which these devices respond to changes in the composition of the surrounding atmosphere is frequently too slow for a range of applications, in which “real time” measurements of oxygen are desired.

Specific areas of sensor need are in the rapid, real-time measurement of fluids and gases, in which composition may be rapidly changing. These can be found in industrial, defense, or medical applications such as blood, saliva, and other liquids associated with the human body including those accessible via a catheter such as the bladder, the peritoneal cavity, or the pleural space.

In this context, any optical technology that provides accurate, rapid measurement of oxygen in a small form factor without direct, wired connections to the sensor constitutes a valuable tool. This mode could meet specific measurement needs for which existing sensors are too slow, heavy, large, or power intensive and prohibit entry into consumer-based applications and a range of other environments of interest.

### SUMMARY OF THE INVENTION

The present disclosure provides a sensor, a device or an apparatus comprising electrospun fibers, a method of making the same, and a method of using the same. Such a sensor or apparatus allows accurate measurement of rapidly changing O₂ levels in gas, liquid or any other sample form comprising gas or liquid using electrospun fiber. Such a sensor, an apparatus or a device comprises electrospun fiber (nanofiber) comprising oxygen sensitive molecules (or called a sensing fiber or a fiber component or element), a light source (or called an excitation source) configured to excite the oxygen sensitive molecules, and a detector element configured to detecting radiation emitted from the fiber. In some embodiments, the device further optionally comprises one or more filters between the light source and the electrospun fiber, or between the electrospun fiber and the detector.

Only a small amount of electrospun fiber may be necessary for sensing. Even a single individual fiber with a submicron diameter has this sensing capability. These sensors easily meet the challenge of miniaturization allowing them to easily become wearable or adapted to space-restricted mobile applications, for example, in healthcare, defense, industrial environmental monitoring, or athletic performance.

To best make use of the underlying technology, specific configurations of electro-optical components are required. These configurations efficiently utilize the electrospun fiber's optical characteristics by providing appropriate levels of illumination followed by efficient detection of the wavelengths of interest. Detection may or may not require the use of filters. They also allow for utilization of this technology in applications benefiting from the rapid response of these sensors as well as allowing for miniaturization.

The sensor element itself can be any suitable arrangement or configuration of electrospun fiber containing the desired molecule or combination of molecules selected to facilitate optical measurements or observations used to detect, identify, and/or quantify dissolved or gaseous oxygen in a solution or gas, respectively, that is in contact with sensing electrospun fibers. The arrangement of electrospun fiber could be a single sheet, a tube having open or closed ends, a single electrospun fiber, or a small or large area of electrospun sensing fiber. More than one type of sensor molecule or combination of molecules may be used to quantify oxygen. More than one type of oxygen sensing fiber may be involved. Ratiometric fluorescent sensing in which an electrospun fluorescent emitter insensitive to oxygen or other environmental conditions (temperature, humidity, etc.) can also be utilized.

The electrospun sensor must be brought into contact with the gas or liquid of interest. This may necessitate complete immersion, partial immersion, or immersion of just one side of said sensor. For either gases or liquids a flow-through arrangement may be necessary or advantageous. Sensing may involve analyzing the sensor's response to elucidate the presence of oxygen to identify the analyte as oxygen, to determine the concentration of oxygen, or combinations thereof.

The function and performance of these sensors in this context is dictated by the degree and form of the excitation radiation used to stimulate the desired emission. For specific applications involving low-cost or miniaturized components, light-emitting diodes (LEDs) have advantages in supplying specific wavelengths of interest with minimal supplied power. In some embodiments, suitable radiation sources are LED devices that are a few millimeters or even sub-millimeter in size. As such, LEDs are ideal to the task of integrating small amounts of sensing electrospun fiber within a variety of application environments. Alternate sources of radiation may include white light, light filtered through a variety of filters, and laser modules. These sources may be placed adjacent to, or in close proximity to, or even contacting the sensing fiber or they may be located at some distance. In addition, their output may be transmitted to the sensing fiber using optical fiber, lenses, mirrors, free space, glass or plastic light pipes, or fiber optic optical rod. Sensor performance may be controlled by the distance between the excitation source and the electrospun fiber. Excitation sources can be placed adjacent to or in close proximity to the sensor or may located at a distance at which the as-stimulated emission from the electrospun fiber can still be detected by other devices. Finally, the lifetime of the sensing fiber can be affected by the length of the period of excitation. Discontinuous excitation is preferred to avoid losses in sensitivity that could lead to time-varying sensitivity.

Sensor performance may also be controlled by the distance between the electrospun sensor and the device that quantifies the optical output from the sensor. The output from the sensor may be transmitted to the sensing device using optical fiber, lenses, mirrors, free space, light pipes, or fiber optic optical rod. Because optical sensing technologies often sense only the total photon flux received, the as-stimulated emission of the electrospun fibers may require filtering to remove wavelengths of radiation not relevant to oxygen sensing. Such optical filters may be either band pass, long pass, short pass or some combination thereof allowing exclusion of wavelengths that interfere with the sensing of the wavelengths of interest.

Detection and quantification of this stimulated emission is critical to measuring the amount of oxygen present in the sample environment of interest. Quantitative or semi-quantitative detection of these emissions may take place using a wide variety of detectors. Spectrometers can be used in this application. Spectrometers function using a diffraction grating that separates components of the incoming light into component wavelengths and establishes the intensity of each of those wavelengths. The intensity or lifetime of the wavelength or wavelengths emitted from the electrospun fiber sensitive to the presence of oxygen are of interest. Additional detection technologies that may be used include photodiodes (with or without pre-amplification), avalanche photodiodes, photomultiplier tubes, light emitting diodes (LEDs) and any other device that can detect or quantify the specific wavelength(s) of interest or their time-varying behavior with or without pre-filtration. The output from these sensing devices may or may not be subject to amplification to achieve the sensitivity required by the application.

In some embodiments, the present disclosure provides a device or apparatus, the method of making and the method of using the same.

In one aspect, a device or apparatus is provided. Such a device (or apparatus) comprises a light source configured to provide light, a sensing element, and a detector. The sensing element comprises nanofiber, which comprises at least one oxygen sensitive compound, and is configured to receive the light from the light source and then emit radiation after being excited by the light. The detector is configured to characterize the radiation emitted from the nanofiber to provide a measured concentration of oxygen in the sample. In some embodiments, the device comprises an enclosure defining an opening for introducing a sample for testing. The sensing element is disposed inside the enclosure. The light sources and the detector may be disposed on two different ends of the enclosure.

In some embodiments, the device further comprises at least one optical filter disposed between the sensing element and the light source or the detector. The at least one optical filter may contact the sensing element in some embodiments.

In some embodiments, the device further comprises at least one heating element, which comprises an electrically conductive and optically transparent material, and is disposed in close proximity to or contacting the sensing element. The at least one heating element is configured to be heated to prevent moisture condensation on the sensing element. In some embodiments, the at least one heating element is an optically transparent disk comprising a layer of indium tin oxide (ITO) coating.

In some embodiments, the device further comprises a porous mask disposed outside or inside the enclosure. For example, the porous mask may be made of expanded polytetrafluoroethylene (ePTFE). In some embodiments, the device further includes a porous block disposed at the opening of the enclosure to reduce or prevent exterior light interference.

The pathway for the light or the radiation can be free space. In some other embodiments, the device may further comprise a different pathway for the light from the light source or the radiation emitted from the nanofiber. The pathway may be selected from the group consisting of a lens, an array of lens, an optical fiber, a light pipe, a fiber optic optical rod, and any other suitable pathway and any combination thereof.

In some embodiments, the sensing element comprises two or more types of nanofibers having different oxygen-sensitive compounds and is configured to provide multiple quantification of oxygen simultaneously. The sensing element may also comprises two or more different oxygen-sensitive compounds in one type of nanofiber. In some embodiments, the nanofiber has a core-shell structure including a core comprising at least one oxygen sensitive compound in a first polymer, and a shell comprising a second polymer.

The detector may be a photodiode or a spectrometer. In some embodiments, the detector comprises a preamplifier configured to amplify a signal based on the radiation emitted from the nanofiber.

The enclosure may be made of an opaque material, and the opening is a hole or a slot, which is located above or below the sensing element. The enclosure may be is in any suitable shape such as a tubular shape. The light source and the detector are disposed on two opposite ends of the enclosure.

In another aspect, the present disclosure provides an apparatus, which comprises an enclosure defining an opening for introducing a sample for testing, a light source configured to provide light, a sensing element, a detector, and at least one heating element for locally increasing temperature. The sensing element comprises nanofiber and is disposed inside the enclosure. The nanofiber comprises at least one oxygen sensitive compound and is configured to receive the light from the light source and then emit radiation after being excited by the light. The detector is configured to detect the radiation emitted from the nanofiber and provide a measured concentration of oxygen in the sample. The at least one heating element comprising an electrically conductive and optically transparent coating, and disposed in close proximity to or in contact with the sensing element. In some embodiments, the electrically conductive and optically transparent coating comprises indium tin oxide.

In some embodiments, the apparatus further comprises a porous mask comprising expanded polytetrafluoroethylene (ePTFE), and is disposed on a surface of the enclosure and covering the opening. The apparatus may comprise at least one optical filter disposed between the sensing element and the light source or the detector, and a light pipe as a pathway for the light from the light source or the radiation emitted from the nanofiber. In some embodiments, the apparatus is a mouth guard or similar device sized and shaped to be wearable by a human subject such as an athlete or a patient, and can be used for directly testing the oxygen content, and indirectly testing the content of carbon dioxide, in the breath of such a human subject.

In another aspect, the present disclosure provides a method of making the device or apparatus as described above. Each component is provided or formed, and then assembled together to provide the device or apparatus.

In another aspect, the present disclosure provides a method of using the device or apparatus as described above. In some embodiments, such a method comprises a step of introducing a sample being a gas or a liquid adjacent to the opening of the enclosure so as to contact the sample with the sensing element. The method further comprises steps of exciting the nanofiber using the light from the light source continuously or discontinuously, and detecting the radiation emitted from the nanofiber so as to provide a measured concentration of oxygen in the sample. In some embodiments, the nanofiber is excited discontinuously in a pulsed mode. In some embodiments, the method comprises heating at least one heating element disposed in close proximity to or in contact with the sensing element to prevent moisture condensation. The at least one heating element comprises an electrically conductive and optically transparent coating as described. The method may further include any other steps as described herein and any step of using the component as described herein.

Additional advantages of the invention will be set forth in part in the description which follows, and in part will be understood from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The present disclosure is best understood from the following detailed description when read in conjunction with the accompanying drawings. It is emphasized that, according to common practice, the various features of the drawings are not necessarily to scale. On the contrary, the dimensions of the various features are arbitrarily expanded or reduced for clarity. Like reference numerals denote like features throughout specification and drawings.

FIG. 1A is an exploded view illustrating exemplary components inside an exemplary apparatus or device in accordance with some embodiments.

FIG. 1B is a cross-sectional view illustrating an exemplary apparatus or device in accordance with some embodiments.

FIG. 1C is a cross-sectional view illustrating the exemplary apparatus or device of FIG. 2 (with a cross-sectional view illustrating the components inside).

FIG. 1D is a cross-sectional view illustrating another exemplary apparatus or device in accordance with some embodiments.

FIG. 2 is an exploded perspective view illustrating an exemplary design of an exemplary device enabling measurement of oxygen in a small form factor in accordance with some embodiments.

FIG. 3 illustrates an exemplary device realized using the exemplary designs of FIG. 2 and any of FIGS. 1A-1D in accordance with some embodiments.

FIG. 4 illustrates an exemplary configuration inside the exemplary device of FIG. 3.

FIG. 5 illustrates electrical output from a photodiode collecting light from the electrospun nanofiber sensor versus the oxygen content of the surroundings in accordance with some embodiments.

FIG. 6 illustrates a front view of an exemplary athletic lip guard (or mouth guard) comprising an exemplary sensor device, for example, as shown in any of FIGS. 1A-1D, in accordance with some embodiments.

FIGS. 7A-7C are perspective views illustrating an exemplary tubular structure of the exemplary sensor device for the exemplary athletic lip guard of FIG. 6.

FIG. 8 is an exploded view illustrating an exemplary sensor device used in the exemplary athletic lip guard of FIG. 6 in accordance with some embodiments.

### DETAILED DESCRIPTION

For purposes of the description hereinafter, it is to be understood that the embodiments described below may assume alternative variations and embodiments. It is also to be understood that the specific articles, compositions, and/or processes described herein are exemplary and should not be considered as limiting.

In the description, spatially relative terms such as “lower,” “upper,” “horizontal,” “vertical,” “above,” “below,” “up,” “down,” “top” and “bottom” as well as derivative thereof (e.g., “horizontally,” “downwardly,” “upwardly,” etc.) may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

In the present disclosure the singular forms “a,” “an,” and “the” include the plural reference, and reference to a particular numerical value includes at least that particular value, unless the context clearly indicates otherwise. Thus, for example, a reference to “an optical filter” is a reference to one or more of such structures and equivalents thereof known to those skilled in the art, and so forth. When values are expressed as approximations, by use of the antecedent “about,” it will be understood that the particular value forms another embodiment. As used herein, “about X” (where X is a numerical value) preferably refers to ±10% of the recited value, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

For the purpose of illustration, some components are depicted using one or more blocks in the drawings.

Reference to “nanofiber” used herein will be understood to cover a fiber having at least one dimension (e.g., diameter) at nanometer level, for example, in the range of 1 nm to 1,000 nm. The other dimension such as length of the nanofiber may be at nanometer, micron or millimeter level.

The present disclosure provides a sensor, an apparatus or a device, a method of making, and a method of using the same. Such a sensor, an apparatus or a device utilizes nanofiber comprising oxygen sensitive molecules to measure local oxygen contents using either discontinuous or continuous excitation followed by detection of the output from the fiber. Such a sensor, an apparatus or a device comprises electrospun fiber (nanofiber) comprising oxygen sensitive molecules (or called a sensing fiber or a fiber component or element), a light source (or called an excitation source) configured to excite the oxygen sensitive molecules, and a detector element configured to detecting radiation emitted from the fiber. In some embodiments, the device further optionally comprises one or more filters between the light source and the electrospun fiber, or between the electrospun fiber and the detector.

The invention relates to allowing measurements of O₂ in gas, liquid or any sample form comprising gas or liquid. As an example, if this focuses on the measurement of O₂ in exhaled air, this would necessarily require an inlet opening for the introduction of exhaled air (typically via a mouthpiece) accompanied by at least one measurement chamber in which the atmosphere will be analyzed and which the sensor unit shown in the drawing must be able to access.

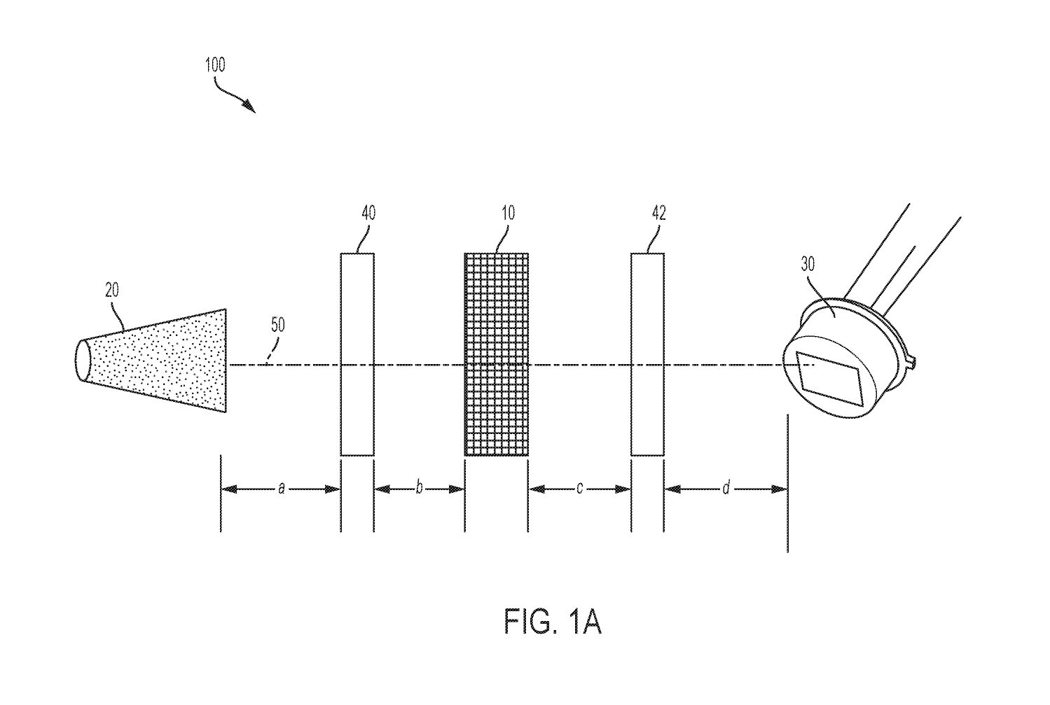

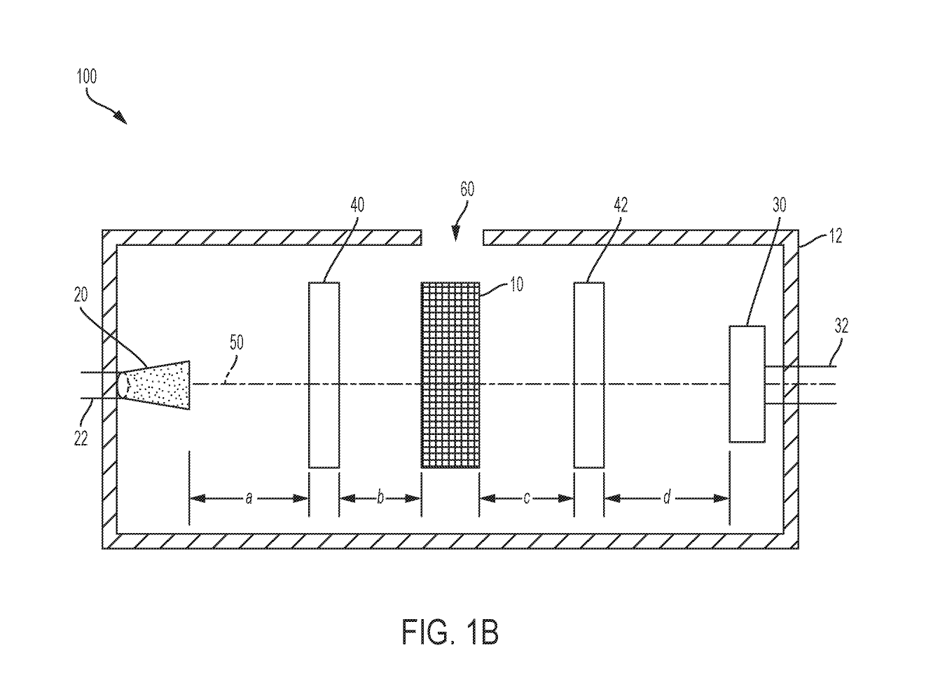

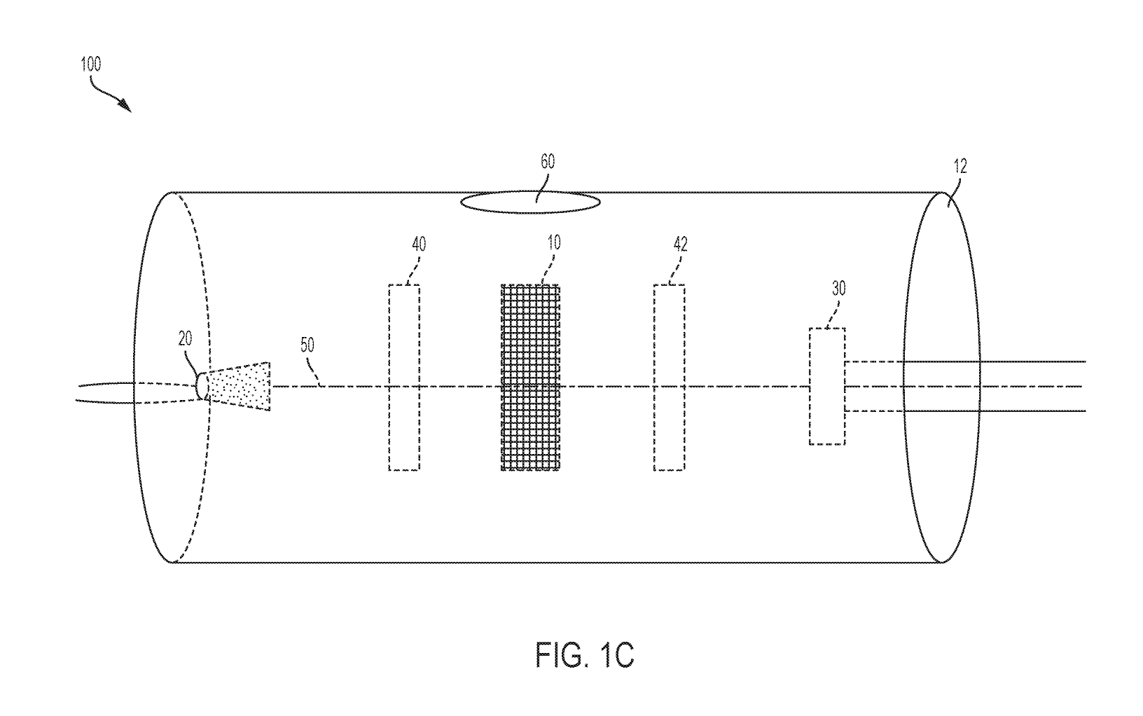

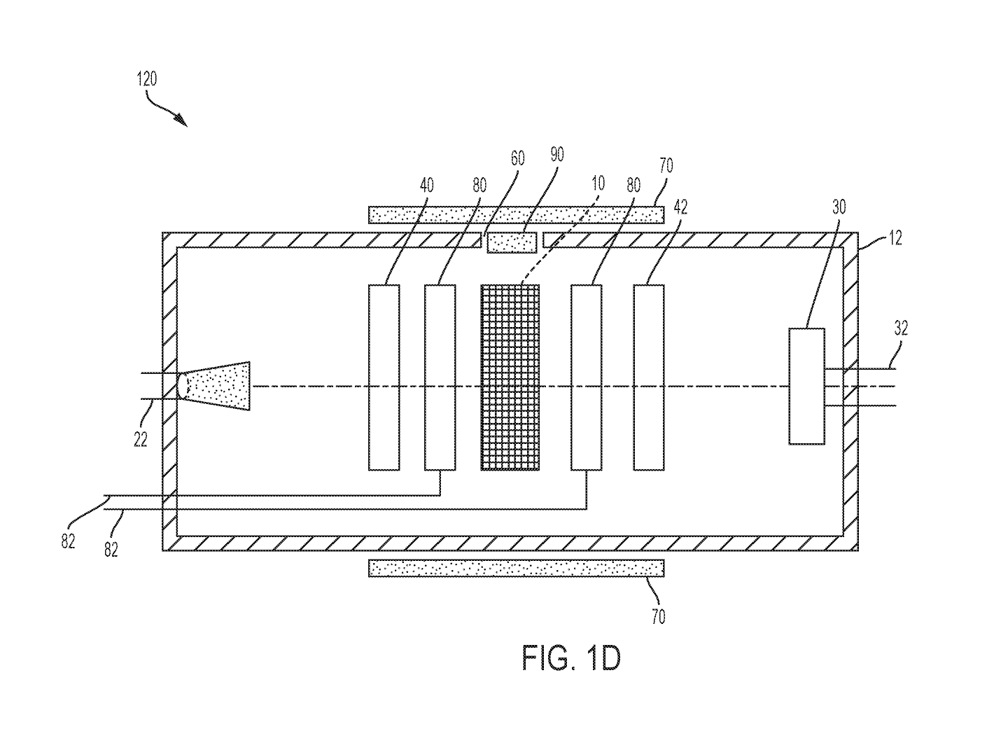

Referring to FIG. 1A, an exemplary device or apparatus 100 is illustrated. The exemplary device (or apparatus) 100 comprises a sensing element 10, a light source 20 configured to provide light, and a detector 30. The sensing element 10 comprises nanofiber, which comprises at least one oxygen sensitive compound. The nanofiber or the sensing element 10 is configured to receive the light from the light source 20 and then emit radiation after being excited by the light. The nanofiber may be electrospun. The detector 30 is configured to detect the radiation emitted from the nanofiber, and provide a measured concentration of oxygen in a sample or surrounding. In some embodiments, the device 100 further optionally comprises at least one optical filter 40, 42, which may include either or both of a first and a second optical filters 40 and 42 as illustrated in FIG. 1A. The first optical filter 40 is disposed between the sensing element 10 and the light source 20. The second optical filter 42 is disposed between the sensing element 10 and the detector 30. The light from the light source 20 and the radiation emitted from nanofiber in the sensing element 10 may travel through the components along a pathway 50 and then reach a surface of the detector 30.

As illustrated in FIG. 1A, the dimensions a, b, c and d represent the distance between the light source 10 and the first filter 40, between the first filter 40 and the sensing element 10, between the sensing element 10 and the second filter 42, and the second filter 42 and the detector 30, respectively. The drawing is not to scale. Each component may be much thinner compared to each of the dimensions a, b, c and d. For example, the sensing element 10 and the optical filter 40, 42 may have a thickness at micron level, while each of the dimensions a, b, c and d is at millimeter level in some embodiments.

The dimensions a, b, c and d can be varied according to the geometric or space needs of the application environment. Light transmission across a, b, c and d can occur via optical fiber, lenses, mirrors, free space, light pipes, or fiber optic optical rod or any other form.

The present disclosure provides an exemplary device 100 (a sensor or an apparatus) that can rapidly measure changes in oxygen concentration in the local environment. The device 100 can be relatively small, less than 5×5×5 mm in size in some embodiments, or may be substantially larger depending on the specific geometric, power and emission needs of the application. The excitation and detection components may be located in the same environment as the sensing nanofiber or may be in a separate environment in which they interact with the sensor by different optical transmission pathways.

The exemplar device 100 comprises one or more of the following components described herein (FIG. 1A), including the sensing element 10, a light source 20, the detector 30, and optical filters 40, 42:

The sensing element 10 comprises electrospun fiber (also referred to as ‘nanofiber’) containing an embedded gas-sensitive compound. The gas-sensitive (e.g., oxygen sensitive) compound or molecule may be present in a weight percentage ranging from about 0.1 wt. % to about 10 wt %, for example, from 0.1 wt. % to 5 wt. %, or from 0.5 wt. % to 3 wt. %. Such a compound may be a luminophore such as a fluorophore. Such a compound is preferably homogeneous dispersed or dissolved in the nanofiber. The nanofiber is optically transparent in some embodiments. The sensing element 10 may be a film made of nanofiber having a thickness at micron level, for example, in the range of from 1 micron (μm) to 100 μm (e.g., from 10 μm to 80 μm).

The gas-sensitive compound may be metal porphyrins or any other suitable molecules. Examples of suitable oxygen-sensitive molecules include, but are not limited to, platinum octaethyl porphine ketone, platinum tetrakis-(pentafluorophenyl)porphin, tris-(4,7-diphenyl-1,10-phenantroline) ruthenium(II) perchlorate, and palladium tetra(pentafluorophenyl)porphyrin. The dispersion of these molecules may be engineered to be as non-agglomerated as possible by control of the pendant groups (e.g., where the Pd atom is surrounded by dendrimeric structures) or by specialized mixing techniques. The fiber itself has nanoscaled characteristics, for example, in the range of 1 nm to 1,000 nm (e.g., 1 nm to 500 nm), in at least one dimension (e.g., diameter), and can be composed of a variety of compositions. One exemplary compound used in some embodiments is a 0.5 wt % loading of platinum(II)-5,10,15,20-tetrakis-(2,3,4,5,6-pentafluorophenyl)-porphyrin contained in a polysulfone core surrounded by a polycaprolactone shell. The associated small diffusion distances provide the advantages of rapid response. This underlying technology allows multiple gas measurements to be recorded in a single second.

In some embodiments, the nanofiber has a core-shell structure including a core comprising the at least one oxygen sensitive compound in a first polymer or glass, and a shell comprising a second polymer or glass. For example, a core comprises a first polymer being oxygen permeable (e.g., silicone, fluorosilicone or polysulfone) and excited molecules in the presence of oxygen and light. The shell, which may be optional, comprises a second polymer, which is optically transparent or biologically compatible. Examples of the fiber having a core-shell structure is described in PCT/US2014/030559 (WO 2014145745 filed Mar. 17, 2014) and the corresponding U.S. Application Publication No. 20160041135, which are incorporated by reference in their entirety. The core may have a diameter in the range of from 1 nm to 1,000 nm, for example, from 100 nm to 600 nm. The shell may have a thickness in the range of from 1 nm to 1,000 nm, for example, from 100 nm to 800 nm.

In some embodiments, the sensing element 10 comprises two or more types of nanofibers having two or more different oxygen-sensitive compounds and is configured to provide multiple quantification of oxygen simultaneously. The sensing element 10 may also comprises two or more different oxygen-sensitive compounds in one type of nanofiber. For example, a first compound such as palladium tetra(pentafluorophenyl)porphyrin can be used to detect a low oxygen content (e.g., 1%-3%). Platinum tetrakis-(pentafluorophenyl)porphin can be used to detect a high oxygen content (e.g., 3% to 10%). The sensing element 10 comprising both compounds can be used to provide multiple quantification of oxygen in different ranges with high sensitivity.

The light source 20 provides light to excite the O₂-sensitive molecule, which emits radiation. The intensity of emitted radiation correlates with the amount of the gas of interest present in the environment. As shown in FIG. 1A, this light source 20 may or may not be filtered to create the impingement of a more desirable wavelength on the sensing nanofiber. A variety of light sources 20 can be employed. The preferred embodiment is a low-cost, low power, light emitting diode (LED) emitting at a wavelength or wavelengths specifically exciting the embedded gas-sensitive molecule. Or it may be a laser that emits a more defined wavelength to achieve the same purpose. The characteristics of this excitation light may be either continuous in nature or pulsed using specific time intervals that avoid the well-known phenomenon of photobleaching of the sensing molecule. Discontinuous excitation is preferred in some embodiments. For example, the light can be applied in a pulsed mode alternating with a period of “on” time (e.g., in the range of from 1 milliseconds to 30 milliseconds) followed by a period of “off” time (e.g., in the range of from 1 milliseconds to 30 milliseconds).

The detector 30 (or detecting element) captures and quantifies the wavelengths of interest or their time-variant behavior. The size of these detecting elements may vary. Miniaturized versions of these elements are preferred in many of the intended application environments. In some embodiments, the detector 30 is placed close to the sensing element 10. In some other embodiments, light may be transmitted to the detector 30 (detector array) using optical fiber, lenses, mirrors, free space, light pipes, or fiber optic optical rod or some other form of light transmission. These methods of light transmission may or may not require precise alignment with the radiation emerging from the sensing element 10.

The light from the light source 20 may have a suitable wavelength in the visible range (e.g., 400-700 nm) or in the ultraviolet range (e.g., 100 nm-400 nm). In some embodiments, a near UV light having a wavelength in the range of from 300 nm to 400 nm is used. The radiation emitted from the nanofiber for detection may have a wavelength in the range of from 500 nm to 700 nm (e.g., 550 nm to 650 nm).

The detector 30 may be a photodiode or a spectrometer. A photodiode measures the overall intensity of the radiation emitted from the nanofiber and reaching the detector 30. A spectrometer measures the intensity of the radiation at different wavelengths. In some embodiments, the detector 30 comprises a preamplifier (not shown) configured to amplify a signal based on the radiation emitted from the nanofiber.

Optical filter 42 may be used to decrease the amount of radiation reaching the detector 30 having wavelengths not germane to measurement of oxygen. The inclusion of a filter element can allow the use of lower cost light sources and detectors appropriate to widespread consumer applications in personal health, industrial environments, or athletic performance monitoring.

The pathway 50 for the light or the radiation can be free space between the components. In some other embodiments, the device 100 may further comprise a different pathway in physical form for the light from the light source or the radiation emitted from the nanofiber. The pathway 50 may be selected from the group consisting of a lens, an array of lens, an optical fiber, a light pipe, a fiber optic optical rod, and any other suitable pathway and any combination thereof.

Referring to FIGS. 1B and 1C, the exemplary device 100 may comprise an enclosure 12 defining an opening 60 for introducing a sample for testing. The sensing element 10 is disposed inside the enclosure 12. The light sources 20 and the detector 30 may be disposed on two different ends of the enclosure 12. Each of the light sources 20 and the detector 30 may be connected with a power source with wires 22 and 32, respectively, which extended outside the enclosure 12.

The enclosure 12 may be made of an opaque material, and the opening 60 is a hole or a slot, which is located above or below the sensing element 10. The enclosure 12 may be is in any suitable shape such as a tubular shape as illustrated in FIG. 1C or a rectanguloid shape. The light source 10 and the detector 30 are disposed on two opposite ends of the enclosure 12.

Referring to FIG. 1D, an exemplary device or apparatus 120 is illustrated. In some embodiments, the device 100 or 120 further comprises a porous mask 70 (or shield) disposed outside or inside the enclosure 12 for prevent moisture from getting into the enclosure 12. For example, the porous mask 12 may be made of expanded polytetrafluoroethylene (ePTFE) or any other hydrophobic material.

In some embodiments, the device 100 or 120 further comprises at least one heating element 80, which comprises an electrically conductive and optically transparent material, and is disposed in close proximity to or contacting the sensing element 10. The at least one heating element 80 is configured to be heated to prevent moisture condensation on the sensing element 10. In some embodiments, the at least one heating element 80 is an optically transparent disk comprising a layer of indium tin oxide (ITO) coating. In some embodiments, the at least one heating element 80 includes two heating elements on both sides of the sensing element 10 as shown in FIG. 1D. The at least one heating element 80 may be connected with a power source through wires 82.

In some embodiments, the device 100 or 120 further comprises a porous block 90 such as a foam disposed at the opening 60 of the enclosure 12 to reduce or prevent exterior light interference. The porous block 90 may be 3D printed plastics having a dark color (e.g., black). Examples of a suitable material for the porous block 90 include polytetrafluoroethylene (PTFE) or any other fluoropolymer.

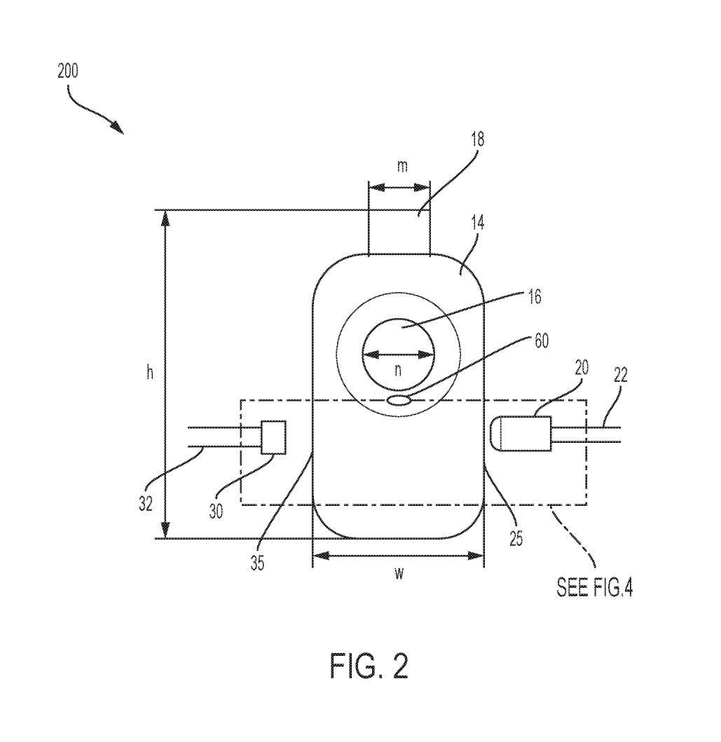

Referring to FIGS. 2-4, an exemplary device 200 is illustrated. The exemplary device 200 enables measurement of oxygen in a small form factor in accordance with some embodiments. FIG. 2 shows schematic view of a device, in which an LED is used as a light source 20, and the detection component (detector) 30 is a photodiode. The filters 40, 42 and the sensing element 10 including electrospun fiber, and the detector 30 are contained within the sensor body 14 and are not visible in FIG. 2. Electrospun fiber is contained within a tube in the body 14 of the device. The tube defines a port (a hole) 60 for allowing access to captured gas streams. An LED is depicted on the right-hand side of FIG. 2 as an excitation component configured to excite an emission from the sensing element 10, which is an electrospun fiber sensor.

As illustrated in FIG. 2, the sensor body 14 may define a cavity or port 16 having a diameter (n), and include a top portion 18 with a width (m). The sensor body 14 further defines a chamber having two exits 25, 35, through which the light source 20 and the detector 30 can be disposed inside the chamber of the sensor body 14. The device 200 may have an overall height (h) and a width (w), each of which may be in the range of from 1 mm to 100 mm (e.g., from 1 mm to 5 mm or from 10 mm to 50 mm).

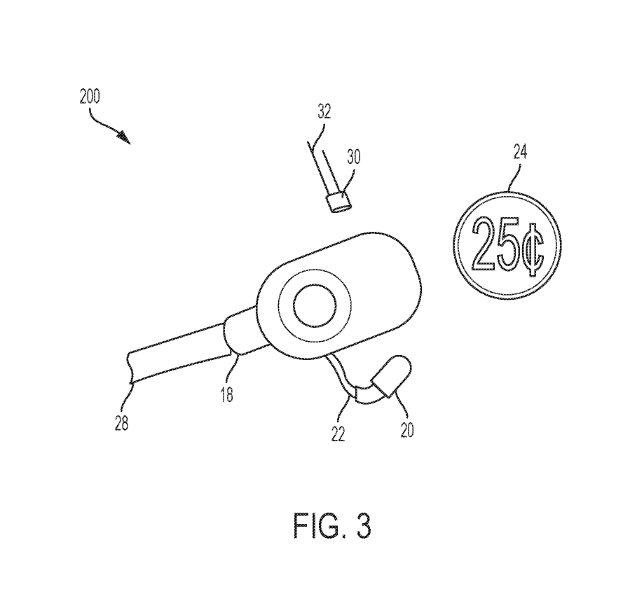

FIG. 3 illustrates an exemplary device 200 realized using the exemplary design of FIGS. 1 and 2 in some embodiments. An LED is visible on the lower side of the sensor body. A photodiode is visible on the upper side of the sensor body. A US quarter 24 is shown as a reference. FIG. 3 illustrates an exemplary assembled sensor structure shown in FIG. 2. An LED and photodiode are shown as components directly inserted into the sensor body making optical contact with the electrospun fiber sensor. A metal conduit (upper left quadrant of image) as the top portion 18 both supplies power and transmits the information from the photodiode externally.

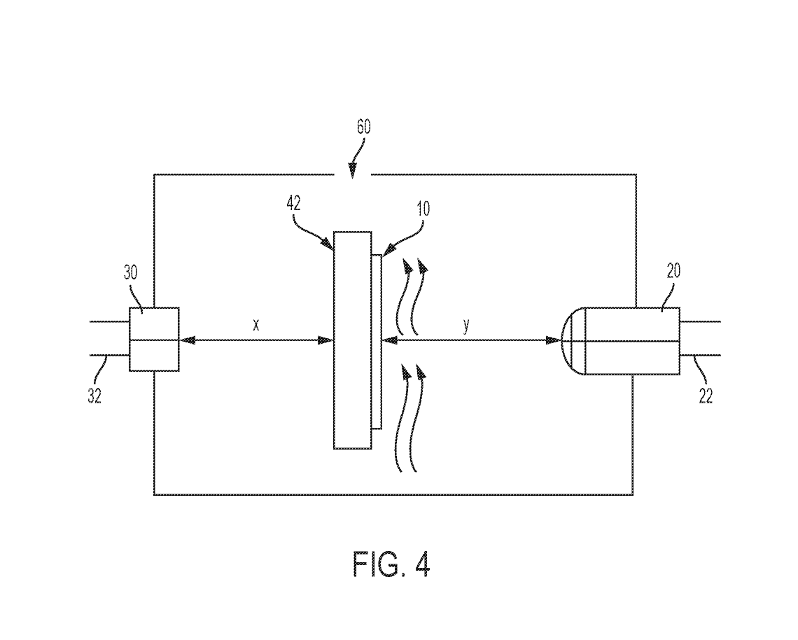

FIG. 4 illustrates an exemplary configuration inside the exemplary device of FIG. 3. FIG. 4 shows how the components of the device can be used to monitor the oxygen content of a gas or liquid passing over the nanofiber sensor. The nanofiber sensor can be either disposable or permanent if protected from contamination. The exemplary configuration in FIG. 4 can be also used for any device in accordance with some embodiments.

Referring to FIG. 4, an exemplary design for the exemplary device 200 is illustrated. Any design as shown in FIGS. 1A-1D can be also used. The exemplary device 200 comprises an LED as the light source 20 supplying excitation wavelengths, an optical filter 42 that selects for a more specific wavelength or set of wavelengths, the nanofiber-based sensor as the sensing element 10, and a photodiode as the detector 10 that quantifies the output from the nanofiber sensor. The wavy vertical line denotes the passage of a gas or liquid containing oxygen that is quantified by the sensing element 10. The optical filter 42 may contact the sensing element 10 in some embodiments. The distance between the sensing element 10 and the light source 20 or the detector may be in the range of from 0.5 mm to 5 mm in some embodiments.

The exemplary device 200 may also include a heating element 80 as described. The light from the LED is captured by a light pipe and transmitted to a transparent disk coated with a thin, transparent indium tin oxide (ITO) layer, which is a heating element 80 (FIG. 1D, not shown in FIG. 2). The ITO layer is electrically heated slightly to eliminate or minimize condensation from moisture-laden human breath. The excitation light passes from the ITO layer into the nanofiber sensor layer itself. There, the light excites an emission in which the intensity and time varying characteristics correlates with the amount of oxygen in contact with the nanofibers. The emitted light leaves the nanofiber, passes through another heating element (or layer) 80 and into another light pipe. The light pipe transmits the optical signal to an optical filter which removes excitation and other wavelengths and enriches the result in the oxygen-sensitive emission. The optical transmission through the filter passes into a photodiode that begins the process of quantification and characterization of the oxygen-sensitive emission. A photodiode is a detection component that receives the output from the electrospun fiber sensor.

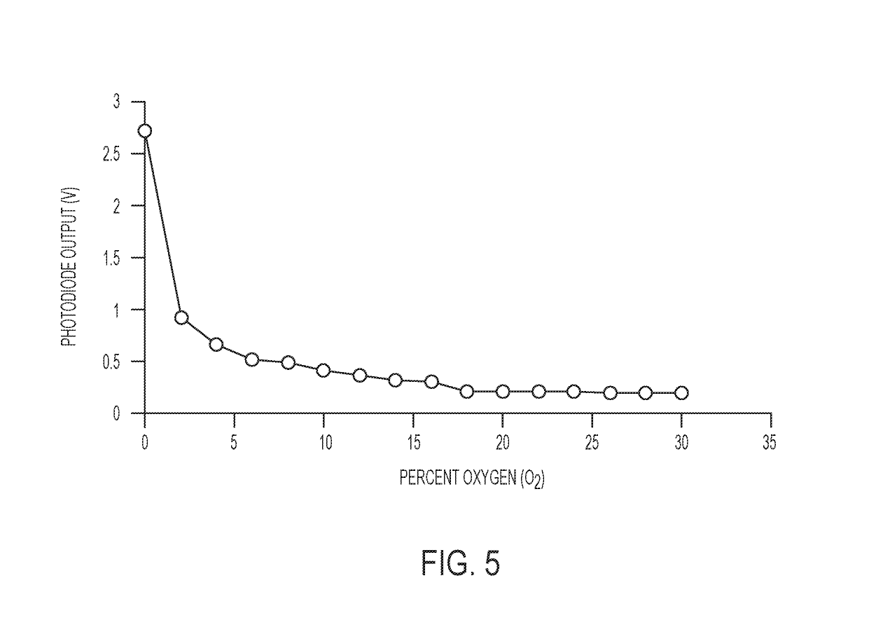

FIG. 5 illustrates electrical output from a photodiode collecting light from an electrospun nanofiber sensor versus the oxygen content (vol. %) of the surroundings in accordance with some embodiments. The wavelength of excitation light used was about 390 nm. The wavelength of emission radiation for measurement was about 670 nm. A lookup table as shown in Table 1 connects the electrical output to the oxygen content experience by an exemplary sensor. A similar output relating emission characteristics (e.g., lifetime) to oxygen content can also be generated. Such lookup tables can be used to connect photodiode output to the oxygen content experienced by the oxygen sensor. In some embodiments, the light excited the oxygen sensitive molecules to emit radiation while oxygen quenches the excited such an emission. Thus, the signal intensity decreases with increasing oxygen concentration.

TABLE 1

| O₂ (vol. %) | Electrical Output (volts) |
|---|---|
| 0 | 2.7 |
| 2 | 0.9 |
| 4 | 0.65 |
| 10 | 0.50 |
| 8 | 0.47 |
| 10 | 0.40 |
| 12 | 0.35 |
| 14 | 0.30 |
| 16 | 0.29 |
| 18 | 0.20 |

The output of the detector 30 component providing a measurement of oxygen is then communicated out of the detector 30 to the external environment. This may occur via wired transmission and/or by wireless (Bluetooth technology standard for exchanging data over short distances using short-wavelength UHF radio waves in the ISM band from 2.4 to 2.485 GHz; Wi-Fi or WiFi technology for wireless local area networking with devices based on IEEE 802.11 standards; or cellular-based communications) communication. This data can either be processed locally, ported to a data processing device (hub or router) or passed along to the Cloud or some other monitoring technology.

For more slowly changing gas concentrations, data processing may include examination of the slope of the data output to determine the direction and degree of change in the gas content (% O₂ or mm O₂). For more rapidly changing analysis, data processing may include manipulation of the peaks and valleys of the data to create a single value measurement of average O₂ content (% O₂ or mm O₂) or instead may report a more detailed description of the time-varying O₂ content.

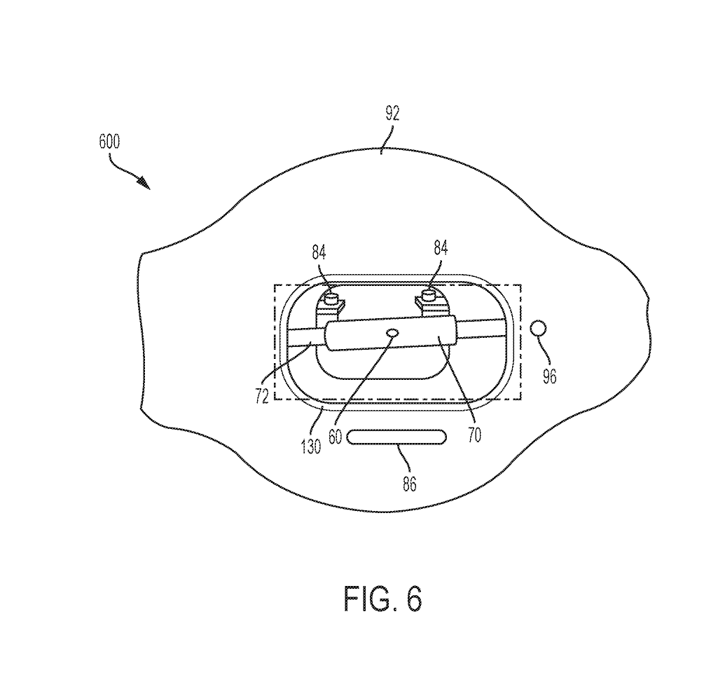

FIGS. 6-8 illustrates another exemplary apparatus 600 in accordance with some embodiment. The shape of the exemplary apparatus 600 in FIGS. 6-8 is for illustration only. The exemplary apparatus 600 may have different shape and design adapted for specified applications. In addition to the design illustrated in FIGS. 6-8, any design or arrangement in FIGS. 1A-1D and 2-4 can be also used.

Referring to FIG. 6, in some embodiments, the exemplary apparatus 600 is a mouth guard (or called a lip guard) or similar device, which is sized and shaped to be wearable by a human subject such as an athlete or a patient. The exemplary apparatus 600 can be used for directly testing the oxygen content, and indirectly testing the content of carbon dioxide, in the exhaled breath of such a human subject. The exemplary apparatus 600 may include auxiliary structures 84, a slot 86, and a main cover 92, each of which is sized and shaped to fit with a regular human subject's mouth. A sensor portion 130 is disposed in a main opening defined by the main cover 92.

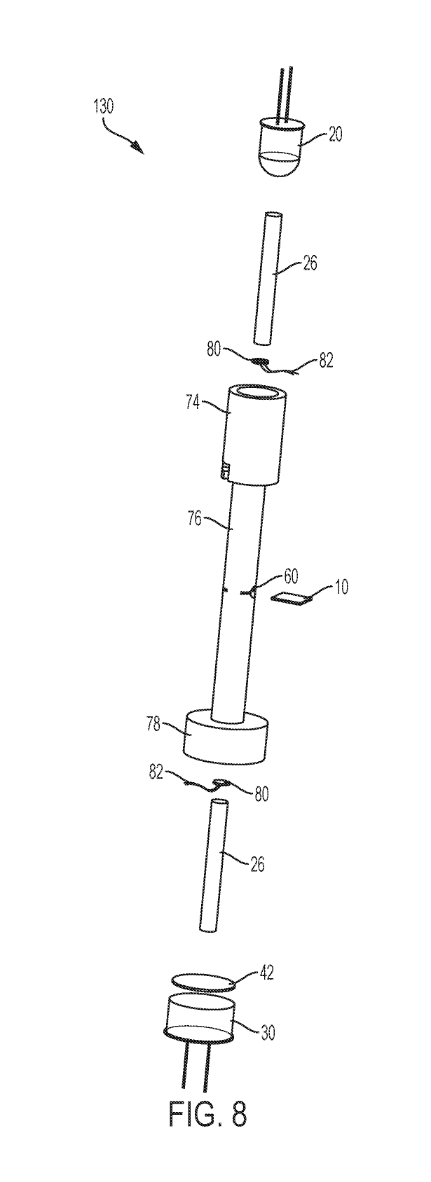

The exemplary apparatus 600 comprises an enclosure 12 defining an opening 60 for introducing a sample for testing. The enclosure 72 in FIGS. 6-8 is one example of the enclosure 12 as described and reference numerals 12 and 72 can be used interchangeably. The enclosure 72 houses the components for the sensor portion 130 described in FIG. 8. Any design or arrangement in FIGS. 1A-1D and 4 can be also suitable for the sensor portion 130. For example, another suitable sensor portion 130 is the exemplary device 120 as described in FIG. 1D. The exemplary apparatus 600 comprises a sensing element 10, a light source 20, a detector 30, and optionally at least one heating element 80 for locally increasing temperature.

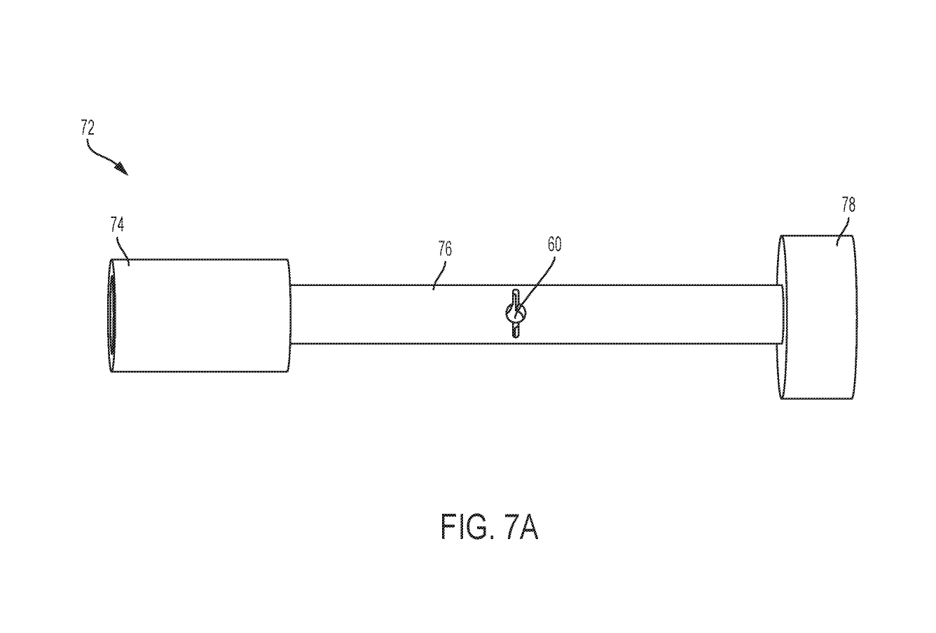

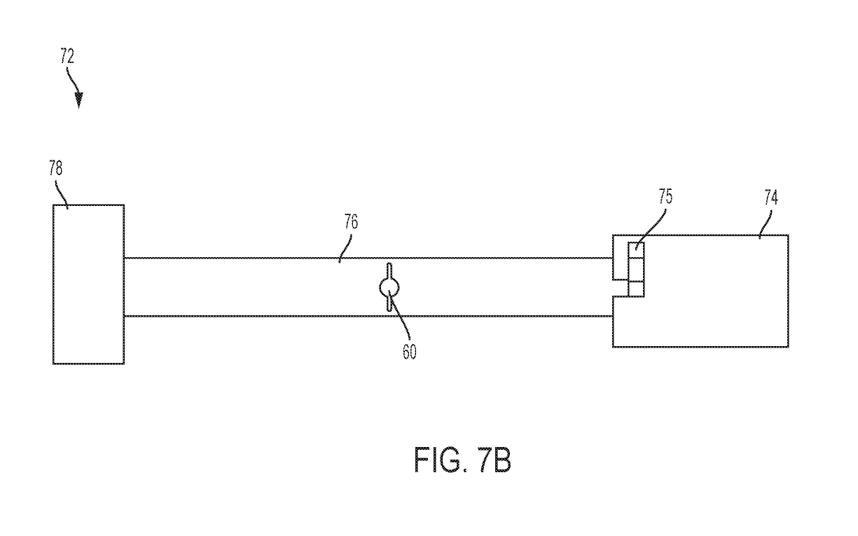

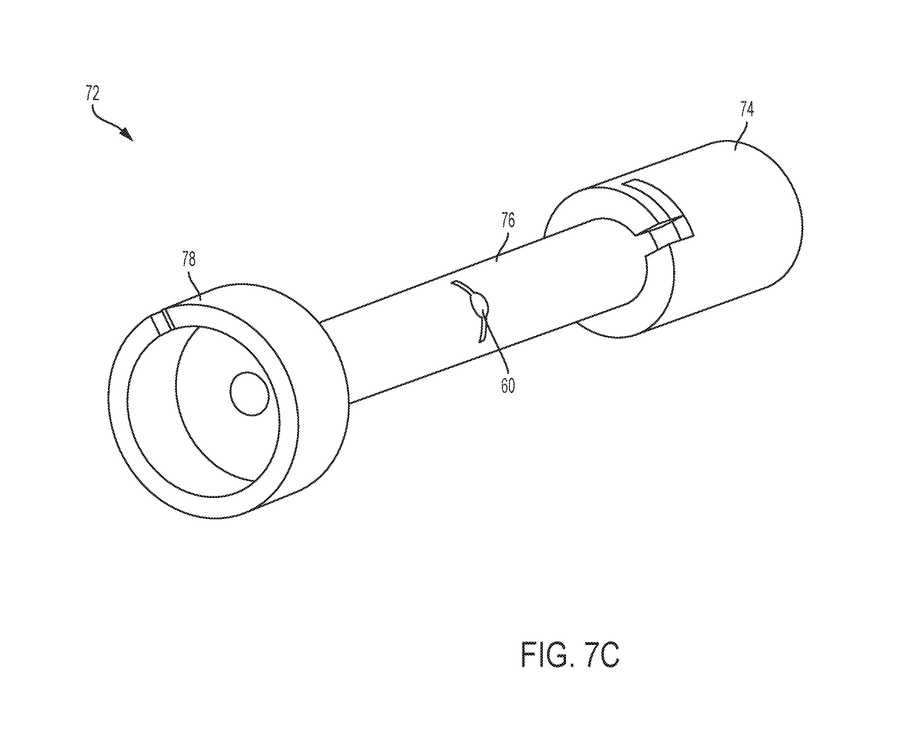

Referring to FIGS. 7A-7C, in some embodiments, the enclosure 72 comprises a first end portion 74 for housing the light source 20, a middle portion 76 for housing the sensing element 10 (and other components such as the heating element 80), and a second end portion 98 for housing the detector 30. The opening 60 may be a hole, a slot, or a combination thereof. Each end portion may include a pin 75 rendering the corresponding end portion removable.

Referring to FIG. 8, the exemplary apparatus 600 comprises a sensing element 10, a light source 20, a detector 30, and at least one heating element 80, for example, two heating elements 80. The light source 20, the detector 30 and the heating element 80 are connected with corresponding power sources through wires. The sensing element 10 comprises nanofiber and is disposed inside the enclosure 72. The nanofiber comprises at least one oxygen sensitive compound and is configured to receive the light from the light source 20 and then emit radiation after being excited by the light. The oxygen may quench the emitted radiation. The detector 30 is configured to detect the radiation emitted from the nanofiber and provide a measured concentration of oxygen in the sample. The at least one heating element 80 comprising an electrically conductive and optically transparent coating, and disposed in close proximity to or in contact with the sensing element 10. In some embodiments, the electrically conductive and optically transparent coating comprises indium tin oxide or any other suitable material.

Referring to FIG. 6, in some embodiments, the apparatus 600 further comprises a porous mask 70 comprising a suitable material, for example, expanded polytetrafluoroethylene (ePTFE), and is disposed on a surface of the enclosure 72 and covering the opening 60. An expanded polytetrafluoroethylene layer covers the aperture to minimize moisture egress to the nanofiber sensor layer.

The apparatus 600 may comprise at least one optical filter 40, 42 disposed between the sensing element 10 and the light source 20 or the detector 30, and a light pipe 26 as a pathway for the light from the light source 20 or the radiation emitted from the nanofiber.

In some embodiments, the enclosure 72 (e.g., a tube) crosses the exhalation port of an athletic lipguard. An LED emission is visible in the small aperture 60 and/or an indicator opening 96 on the cover 92.

An example of oxygen monitoring would be breath-by-breath analysis. Given the nature of the nanofiber sensor, inhalation at atmospheric oxygen levels would correspond to ‘valleys’ in the data (in which the emission of the sensing molecule is relatively quenched) alternating with ‘plateaus’ corresponding to exhalations (in which the emission of the sensing molecule is enhanced by the presence of less oxygen). The valleys in the data could provide dynamic information regarding the amount of oxygen being inhaled, which can be important in either calibration or specific industrial, defense or medical applications. The plateaus in this data correspond to measurements of how much oxygen is present in the exhalations. For example, in application to athletic performance this can be used to quantify metabolic efficiency. For an application in a cockpit, this is a measure of potential for incipient hypoxia (indicated by too-low levels of either inhaled or exhaled O₂). In industrial applications, if the air sampled by the nanofiber sensor has a too-low oxygen content, this could indicate a potentially hazardous environment control problem. For a medical application, too-low levels of exhaled oxygen may be indicative of congestive heart failure.

The sensor element 10 itself can be any arrangement of electrospun fiber having the desired molecule or combination of molecules selected to facilitate optical detection, identification and/or quantification of oxygen in a sample in contact with the electrospun fibers. For example, the sensing element 10 comprising nanofiber may be in the form of a sheet, a tube having open or closed ends or a single electrospun fiber or small number of electrospun fibers. More than one type of sensor molecule or combination of molecules may be used to quantify multiple substances simultaneously. More than one type of sensing fiber may be present, meaning that the sensor itself may contain multiple compositions of sensing fiber. The presence of a compound, quantum dot or some other condition-insensitive moiety may be included to provide a comparison allowing ratiometric sensing of one or more compounds of interest.

The sensor can be brought into close contact with the analyte of interest. This may necessitate complete immersion, partial immersion, or immersion of just one side of said sensor. For gases or liquids a flow-through arrangement may be necessary or desirable. Sensing may involve analyzing the sensor's response to elucidate the presence of oxygen, to identify the presence of oxygen, to determine the concentration of oxygen or combinations thereof.

An additional embodiment is as a thin layer of electrospun fiber on one side of a window that is sufficiently optically transparent to both the excitation and the emission wavelengths. This could then allow creation of a closed end probe that could be inserted into the environment of interest.

The function and performance of these sensors in this context is dictated by the degree and form of the excitation radiation used to stimulate the desired emission. For specific applications involving low-cost or miniaturized components light-emitting diodes (LEDs) have very well described advantages in supplying specific wavelengths of interest. In some embodiments, LEDs used are in multi-millimeter or sub-millimeter dimensions ideal to the task of integrating small amounts of sensing electrospun fiber within a variety of environments. Additional sources of radiation may include white light, light filtered through a variety of filters, and laser modules such as the 405 nm 5 mW 12×30 mm Laser Module sold by AixiZ OEM Electronics. This light may be transmitted to the detector array using optical fiber, lenses, mirrors, free space, light pipes, or fiber optic optical rod or some other form of wavelength transmission. These methods of light transmission may or may not require precise alignment with the radiation emerging from the sensor.

The as-stimulated emission may require filtering to remove wavelengths of radiation not of interest to oxygen sensing. Plastic sheet filters such as the CL182 Cool LED Light Red Gel Filter (Lee Filters Inc.) are used in some embodiments. Alternatively, a more expensive band-pass filter may be used to pass frequencies within a certain range and reject (attenuate) frequencies outside that range. Alternatively, an interference filter may be used to reflect one or more spectral bands or lines and transmit others while maintaining a nearly zero coefficient of absorption for all wavelengths of interest. Simple colored long pass glass filters may also be sufficient to remove wavelengths that might interfere with sensing having the desired efficiency.

Sensor performance is partly controlled by the distance between the excitation source and the electrospun fiber. Excitation can be placed immediately adjacent to the sensor or may be located at a distance in which as-stimulated emission from the electrospun fiber can still be detected. Optical fiber interrogation is particularly valuable as a means of retrieving information as either single optical fibers or fiber bundles can serve as both a pathway for excitation wavelengths as well as those wavelengths emitted by the sensor in response.

Detection of this stimulated emission provides information quantifying the amount of oxygen present in the sample of interest. Quantitative or semi-quantitative detection of these emissions make take place using a wide variety of detectors. Spectrometers are used in some embodiments. The lifetime of the emission present an alternative method of establishing lifetime. Spectrometers function using a diffraction grating that separates components of the incoming light into component wavelengths and establishes the intensity of each of those wavelengths. The intensity or time varying characteristics of that wavelength emitted from the electrospun fiber is sensitive to the presence of oxygen. Additional detection technologies that may be used include photodiodes (with or without preamplification), photomultiplier tubes, RGB (tricolor including red, green and blue) LEDs, phototransistors or any other device that can detect or quantify the wavelength(s) of interest or their time-varying behavior.

In another aspect, the present disclosure provides a method of making the device or apparatus (e.g., 100, 120, 200, 600) as described above. Each component is provided or formed, and then aligned and assembled together to provide the device or apparatus.

In another aspect, the present disclosure provides a method of using the device or apparatus (e.g., 100, 120, 200, 600) as described above. In some embodiments, such a method comprises a step of introducing a sample being a gas or a liquid adjacent to the opening of the enclosure 12 (or 72) so as to contact the sample with the sensing element. The method further comprises steps of exciting the nanofiber using the light from the light source 20 continuously or discontinuously, and detecting the radiation emitted from the nanofiber so as to provide a measured concentration of oxygen in the sample. In some embodiments, the nanofiber is excited discontinuously in a pulsed mode. In some embodiments, the method comprises heating at least one heating element 80 disposed in close proximity to or in contact with the sensing element to prevent moisture condensation. The at least one heating element 80 comprises an electrically conductive and optically transparent coating as described. The method may further include any other steps as described herein and any step of using the component as described herein.

In some embodiments, the oxygen can be determined within a gas sample directly contacting the electrospun fiber in the sensing element 10. The gas sample can be, for example, an environmental sample (e.g., an air quality sample), a process gas, an emission gas (e.g., in an industrial setting or exhaled mammalian breath), or a gaseous fuel stream.

In some embodiments, the oxygen can be determined within a liquid sample directly contacting the electrospun fiber in the sensing element 10. The liquid sample can be, for example, an aqueous solution, such as a wastewater sample or a liquid biological sample. In some embodiments, the sample can comprise a biological sample (e.g., perspiration, a solution or suspension of cells, an in vitro cell or tissue culture, or tissue in vivo). In some embodiments, the sample can comprise a bodily fluid. “Bodily fluid,” as used herein, refers to a fluid composition obtained from or located within a human or animal subject. Bodily fluids include, but are not limited to, urine, ascites, whole blood, pleural effusion, blood plasma, peritoneal fluid, serum, tears, semen, saliva, sputum, exhaled breath, nasal secretions, pharyngeal exudates, bronchioalveolar lavage, tracheal aspirations, interstitial fluid, lymph fluid, meningal fluid, amniotic fluid, glandular fluid, feces, perspiration, mucous, vaginal or urethral secretion, cerebrospinal fluid, and transdermal exudate. Bodily fluid also includes experimentally separated fractions of all of the preceding solutions, as well as mixtures containing homogenized solid material, such as feces, tissues, and biopsy samples.

An additional area of biomedical application is in vivo oxygen sensing and measurement of oxygen tension in a variety of biomedical environments. Minimally invasive measurements can take place by virtue of a small electrospun fiber-based sensor probe placed into the end of a catheter. In this position, this sensor has access to fluids in or around the target organ. For example, this could be the bladder, the peritoneal space around the heart or the pleural space around the lung. Fiber optics may be used to excite and interrogate a fiber-based sensor located at the end of such a catheter. Alternatively, a small source+detector package could be located at the end of the catheter and information regarding the local oxygen concentration transmitted to the external environment either through a wire or wirelessly.

If used to examine exhaled breath or other multi-component gases, a gas conditioning unit, a device for air dehumidification, for filtering respiratory aerosols, a filter or additional devices could be located upstream to condition the exhaled air. Through the application of thermal insulation such gas sensors could be operated or maintained at their respective optimum temperatures. As a calibration, external air could be supplied through a filter such as activated carbon.

For measurement of exhaled breath a temperature and a humidity sensor may be located within the sample chamber or enclosure. If necessary, these additional sensors would allow for corrections with respect to temperature and water vapor partial pressure to thereby take place. In addition, temperature, humidity and pressure sensors located outside the sample chamber may also be necessary to ensure accuracy.

These sensors may or may not require a porous shield (such as porous fluoropolymer) to protect the sensor from direct exposure to specific elements in the gas or fluid stream while enabling continued accurate measurements of oxygen. Expanded polytetrafluoroethylene is preferred in some embodiments due to its inherent hydrophobicity that prevents passage of physical water or hydrophilic contaminants while allowing oxygen, water vapor and other common gases to pass. Expanded polytetrafluoroethylene is available from W. L. Gore and Associates of Delaware, USA, under trademark of GORE-TEX®. Similarly, a hydrophilic membrane could be used to allow the passage of water from aqueous suspensions while restricting the flow of contaminants. In some embodiments, localized heating of the optical components or their surfaces is used to prevent condensate in the form of small water droplets that might result in a net decrease in the transmitted optical signal.

A modular measurement platform for respiratory gas analysis allows an increased market potential in which development focuses on making these devices less expensive allowing penetration of a broad consumer market.

This technology can be used for activity tracking associated with the effects of specific activities such as walking, sleeping, exercise and heart rate and food consumption on exhaled O₂ levels to provide individual consumers with immediate and vital feedback upon which they can make informed decisions regarding their own health or athletic performance.

This technology can be used to monitor patient status or a specific diagnosed condition (e.g., idiopathic pulmonary fibrosis or congestive heart failure) in real time to provide faster, more accurate notification of physiological irregularities than considerably slower pulse oximetry monitoring. Dynamic oxygen sensing provides a more complete picture of potentially rapid changes in patient health.

Soldier systems can benefit as individual O₂ sensing can lead to integrated networks that link soldiers in a larger battlefield and allow remote management and monitoring of individual and aggregate stress levels to predict performance. In this context intra-oral deployment of these sensors (in a mouthguard or artificial tooth, for example) can be used to directly measure oxygen both entering and leaving the oral cavity. Similarly, intra-nasal deployment of these sensors could also be used to directly measure oxygen both entering and leaving the nasal cavity.

By a simple assumption regarding the difference in the amount of oxygen in exhaled breath versus the known amounts contained within the inhaled gas, the amount of CO₂ in exhaled breath can be indirectly determined. This is known to correlate with levels of psychological stress, for example. Faster, shallower breaths associated with stress remove CO₂ from the blood faster than normal. Low CO₂ levels cause increased feelings of anxiety and fear and activate in the sympathetic nervous system, causing the “fight or flight” cycle. By monitoring calculated levels of exhaled CO₂, individuals can restore and maintain blood CO₂ levels to reverse the stress cycle.

Individual athletes can benefit from clear, real-time readout of performance that naturally complements other biometric data such as heart rate and performance characteristics. Employed in integrated teams of athletes, these sensors can allow for rapid identification and assessment of those who might be tired, undernourished, dehydrated or overtaxed in some way.

This technology can also be used to sense or quantify rapidly varying oxygen concentrations in applications relating to the supply of oxygen for applications in fields such as medicine, aerospace, or industry.

Sensing in medical or aerospace environments is of particular importance as these provide critical feedback regarding the state of patients, pilots or underwater divers. For example, idiopathic fibrosis is associated with decreased lung function and progression of this disease would cause increases in exhaled O₂ detected in-mask accompanied by decreases in exhaled CO₂. Pilots undergoing high g maneuvers must perform special breathing exercises to avoid or prevent unconsciousness. Rapid (<1 sec) exhalation/inspiration cycles every 3-4 seconds are required to maintain oxygen content and decrease the levels of carbon dioxide in blood while also relieving increased pressure on the chest allowing the heart to refill with blood. Conversely, divers operating at depth utilizing rebreather technologies often have no reliable, quantitative, real time information regarding the level of inhaled O₂ being provided by their rebreather equipment.

In addition, these sensors can be embedded into garments or fabrics that allow gases or liquids to freely pass to allow simultaneous measurement of the oxygen content of these gases or liquids. An example is flame resistant hoods and balaclavas in which measurement of both inhaled and exhaled gas passing through the fabric by an embedded sensor provides important data regarding the health and status of the wearer.

The photodetector used to sense the emitted radiation from the fiber can exhibit a variety of useful performance parameters. Photodetectors or light sensors can be divided into three broad categories: external photoelectric effects, internal photoelectric effects and thermal types. Photomultiplier tubes (PMTs) make use of the external photoelectric effect and have superior response speed and sensitivity. Light sensors utilizing the internal photoelectric effect can be further divided into photoconductive types and photovoltaic types. Photoconductive cells represent the former, and p-i-n photodiodes the latter. Both types feature high sensitivity and miniature size, making them suited for use as sensors in confined environments. Thermal types have low sensitivity but have no wavelength dependence. An example of the latter is a NIR/RED Enhanced 5 mm² photodiode-preamplifier preamplifier diode (Opto Diode, Inc.) comprised of a low-noise photosensor comprising a Si photodiode, an op amp and feedback resistance and capacitance integrated into the same package. Or it may be an Diffused RGB (tri-color) LED (Adafruit Industries). A spectrometer (for example, from Ocean Optics) can be used to track specific wavelengths dynamically and follow peak values in spite of small changes in position. A phototransistor/ambient light sensor (for example, from Everlight Inc) converting light into current in either photovoltaic, photoconductive or avalanche modes may also be suitable. A regular photomultiplier tube (PMT) or a microPMT (for example, from Hamamatsu Inc) can be employed for highly sensitive, single photon measurements suited to detecting and physically tracking the source of small changes in O₂ in three-dimensional space.

An example of a highly portable spectrometer is Ocean Optic's STS spectrometer and Development Kit which combines a portable, lightweight microspectrometer, a Raspberry PI board, a 3000 mAh battery and a 150 mB/s wifi host to allow for direct readouts to and control by cellular phone platforms.

In addition, using high sensitivity single photon detectors/photomultiplier tubes could possibly allow for the sensing, triangulating and tracking of non-ambient O₂ levels. For example, these could be associated with the presence of human activity in specific locations. This capability could be combined with either remote or unmanned aerial vehicle-mounted sensing to locate and track individuals or groups of individuals. In addition, they could also be used to establish unauthorized levels of industrial O₂ emissions, based on the amount of O₂.

In athletic or military applications such sensor systems are required to be high performing, durable and rugged while also being lightweight. Nanofiber-based sensor systems are extremely durable as the sensor elements themselves can be crushed, dropped or exposed to high g forces with no significant change in efficiency or function. Relative to Raman spectroscopy, fuel cell measurement and other techniques electrospun fiber is more durable, smaller and more reliable and impervious to mechanical shock.

Activity data from a nanofiber-based device or analyzer can be downloaded to an app on either a smartphone or a laptop computer or some other device. Such an app could provide a detailed analysis of trends versus time to track progress. Sensors in medical applications are routinely used to measure and monitor blood flow, pulse, blood pressure, blood oxygen levels, muscle movement, body fat and body weight. The most successful versions of these sensors are those that use algorithms to process the raw, sensed data into actionable, meaningful insight for a given user.

Such sensors must be able to communicate with the larger world. While wireless connectivity through short-range radio or other wireless protocols are popular, wired connectivity through a USB port is also possible.

Many such sensors could be worn during sports or other rugged activities. Ruggedness is defined in terms of the application. Soldier-mounted sensors would operate on an entirely different level of ruggedness, requiring wider temperature ranges, better shock and vibration resistance and resistance to chemicals or solvents that would ruin a consumer device.

Contactless data connections could use transceivers in the device to create a wireless connection. This approach can support high-speed I/O protocols such as USB 2.0 and 3.0. The short distance between transceivers provides a power-efficient connection. Such connected devices can have an IP address allowing them to communicate with every other IP device and join the Internet of Things (IoT) to download activity regarding exhaled O₂ and to an app on a computer, for example, providing a detailed analysis of trends over time to track athletic progress, caloric expenditure and fitness.

Power can be supplied to the sensor (device or apparatus) by a variety of techniques. Wired connections to the grid can be used as well as batteries and capacitors. Batteries can be charged via induction without a direct electrical connection. A variety of energy-harvesting techniques (such as piezoelectric or thermoelectric technologies) can be used to power the sensors. These can provide a trickle of current to charge a battery or a capacitor to allow the device to go longer between charges. A free space antenna could also be used to harvest the necessary energy from the sensor surroundings. Solar cells are an alternative source of energy.

The present disclosure provides a sensor, an apparatus or a device as described. Such a sensor, an apparatus or a device may further comprise any of the components specifically described herein (e.g., fabric or porous mask or coating, pre-amplifier), or any other components further needed, including but are not limited to, a cover, wiring, a power source, and an electric plug.

In some embodiments, more than one type of sensor molecule or combination of molecules is present to provide multiple quantifications of oxygen simultaneously. More than one type of sensing fiber may be present, meaning that the sensor itself may contain multiple compositions of sensing fiber. In some embodiments, the presence of a compound, quantum dot or some other condition-insensitive moiety, which is inert to oxygen, is included to provide for ratiometric sensing of oxygen.

In some embodiments, the sensing fiber may be coated with or embedded in a fabric or porous mask, which may comprise a hydrophilic or hydrophobic material (e.g., a fluoropolymer).

The sensor may be completely immersed, partially immersed, or immersed on just one side. In some embodiments, gases or liquids pass through the sensor fiber itself. In some embodiments, the gases or liquids of interest pass through a fabric or porous mask in which the sensing fiber itself is embedded.

In some embodiments, the sensing fiber exists as a thin layer on one side of a window optically transparent to both excitation and emission wavelengths. The sensing fiber may be disposed adjacent to or in contact with the excitation source, or sits directly on the excitation source (e.g., an LED). In some embodiments, the sensing fiber is disposed adjacent to or in contact with sits directly on, the optical filter. An optical fiber may serve as a pathway for excitation and/or emission wavelengths. A lens or array of lenses may serve as a pathway for excitation and/or emission wavelengths in some embodiments. Free space serves as a pathway for excitation and/or emission wavelengths in some embodiments. A light pipe serves as a pathway for excitation and/or emission wavelengths in some embodiments. A fiber optic optical rod serves as a pathway for excitation and/or emission wavelengths.

In some embodiments, light transmission for excitation requires precise alignment with the pathway for detection on the other side of the sensor. Light transmission for detection may require precise alignment with the pathway for excitation on the other side of the sensor.

Examples of a suitable light source include but are not limited to an LED and a laser. The light source is used to excite the electrospun fiber to measure the oxygen content of its surroundings. In some embodiments, an LED light source is used to excite the electrospun fiber to measure the oxygen content of its surroundings.

In some embodiments, appropriately filtered white light is utilized to excite the electrospun fiber to measure the oxygen content of its surroundings. In some embodiments, a single or multi-layer filter set is used to only allow passage of a specific wavelength or wavelengths emerging from the excitation source to stimulate oxygen measurement of the sensor surroundings. In some embodiments, a single or multi-layer filter set is used to only allow passage of a specific wavelength or wavelengths emerging from the electrospun fiber to be transmitted to the sensing element itself to provide measurement of the oxygen content of its surroundings.

In some embodiments, a photodiode with or without a pre-amplifier is used to detect or quantify emissions from the electrospun fiber to measure the oxygen content of its surroundings. For example, an RGB diode detector is used in some embodiments. A normal, micro- or miniaturized spectrometer can be also used to detect or quantify emissions from the electrospun fiber to measure the oxygen content of its surroundings. In some embodiments, a photomultiplier tube (PMT) is used to detect or quantify emissions from the electrospun fiber to measure the oxygen content of its surroundings. An operational amplifier (e.g., op-amp or opamp) may be also used to produce an output potential larger than the potential difference between its input terminals presented by the sensing unit (such as a photodiode).

In some embodiments, with the electrospun fiber, an appropriate window is used for allowing passage of the necessary wavelengths into a vessel or tube to measure the oxygen content within that vessel or tube. A source and a detector are placed within the vessel or tube to measure the oxygen content within that vessel or tube. In some embodiments, the fluorescence emerging from an exposed layer of oxygen sensitive nanofibers is detected to measure the oxygen content within a vessel or tube.

The method of using the device described herein comprises at least one step of applying a sample to be adjacent to or in contact with the sensing fiber described herein. The method may further comprising other steps such as calibrating the device. The applications is include any application in which oxygen content needs to be measured. The samples can be in the form of liquid, gas, or any form comprising a liquid such as a paste, a suspension or a colloid. This technology can measure the oxygen content of exhaled breath within a vessel, tube, or mask of any kind. This technology can accurately measure breathing rate including hyperventilation, hypoventilation or cessation of breathing within a vessel, tube, or mask of any kind. For another example, this technology can measure the oxygen content in biological fluids.

Other simultaneous measurement can be also made when an oxygen content is measured. In some embodiments, a simultaneous measurement of temperature of the electrospun fibers themselves improves either the accuracy or reliability of dynamic oxygen measurement. In some embodiments, any simultaneous measurement of humidity or water content of the electrospun fibers improves either the accuracy or reliability of dynamic oxygen measurement. In some embodiments, any simultaneous measurement of external temperature improves either the accuracy or reliability of dynamic oxygen measurement. In some embodiments, any simultaneous measurement of external humidity improves either the accuracy or reliability of dynamic oxygen measurement.

In some embodiments, dynamic measurement of oxygen is experienced at the end of a catheter. In some embodiments, dynamic measurement of small variations in oxygen in the ambient air is used as a method of establishing or locating the source or sources of emission.

In some embodiments, a fluoropolymer, for example, expanded polytetrafluoroethylene (ePTFE), or other porous shield can be used to protect the sensing fiber and electronic components from components of the measurement environment.

In some embodiments, the sensor or device is used examining breath to quantify metabolic efficiency using algorithms to process the raw, sensed data into actionable, meaningful insight for a given user. The sensor or device can be also used in examining breath in a team setting to allow for identification and assessment of those individuals who might be tired, undernourished, dehydrated or overtaxed in some way.

In some embodiments, power is supplied to the sensor via wireless techniques such as induction, energy-harvesting techniques, or a free space antenna.

In some embodiments, the sensor or device is deployed intra-orally to directly measure oxygen either entering or leaving the oral cavity, or intra-nasally to directly measure oxygen either entering or leaving the nasal cavity, or in vivo to directly measure oxygen within a specific area of the body.

The device described herein allows utilization of electrospun fiber sensor containing an oxygen sensitive molecule to dynamically measure the oxygen content of surrounding gases or liquids in a variety of environments on a <1 second time scale, using light sources providing either continuous or discontinuous excitation followed by detection and quantification of the light emitted by the sensor.

For one example, the oxygen content for a room or other confined space can be measured dynamically to establish the presence of less-than-optimal or lethally low levels of O₂. The data can be used to trigger increased room demand for air circulation. The the presence of less-than-optimal or lethally low levels of O₂ can trigger a warning system that alerts the wearer and/or a local network of the presence of these hazardous conditions.

The methods and system described herein may be at least partially embodied in the form of computer-implemented processes and apparatus for practicing those processes. The disclosed methods may also be at least partially embodied in the form of tangible, non-transient machine readable storage media encoded with computer program code. The media may include, for example, RAMs, ROMs, CD-ROMs, DVD-ROMs, BD-ROMs, hard disk drives, flash memories, or any other non-transient machine-readable storage medium, or any combination of these mediums, wherein, when the computer program code is loaded into and executed by a computer, the computer becomes an apparatus for practicing the method. The methods may also be at least partially embodied in the form of a computer into which computer program code is loaded and/or executed, such that, the computer becomes an apparatus for practicing the methods. When implemented on a general-purpose processor, the computer program code segments configure the processor to create specific logic circuits. The methods may alternatively be at least partially embodied in a digital signal processor formed of application specific integrated circuits for performing the methods.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A device comprising:
an enclosure defining an opening for introducing a sample for testing;
a light source configured to provide light;
a sensing element comprising nanofiber and disposed inside the enclosure, the nanofiber comprising at least one oxygen sensitive compound and configured to receive the light from the light source and then emit radiation after being excited by the light; and
a detector configured to detect the radiation emitted from the nanofiber and provide a measured concentration of oxygen in the sample.

2. The device of claim 1, further comprising:
at least one optical filter disposed between the sensing element and the light source or the detector.

3. The device of claim 2, wherein the at least one optical filter contacts the sensing element.

4. The device of claim 1, further comprising:
at least one heating element comprising an electrically conductive and optically transparent material, disposed in close proximity to or contacting the sensing element, and configured to be heated to prevent moisture condensation on the sensing element.

5. The device of claim 4, wherein the at least one heating element is an optically transparent disk comprising a layer of indium tin oxide (ITO) coating.

6. The device of claim 1, further comprising:
a porous mask disposed outside or inside the enclosure.

7. The device of claim 6, wherein the porous mask is made of expanded polytetrafluoroethylene (ePTFE).

8. The device of claim 1, further comprising:
a porous block disposed at the opening of the enclosure to reduce or prevent exterior light interference.

9. The device of claim 1, further comprising:
a pathway for the light from the light source or the radiation emitted from the nanofiber, the pathway selected from the group consisting of a lens, an array of lens, an optical fiber, a light pipe, and a fiber optic optical rod.

10. The device of claim 1, wherein the sensing element comprises two or more types of nanofibers having different oxygen-sensitive compounds and is configured to provide multiple quantification of oxygen simultaneously.

11. The device of claim 1, wherein the nanofiber has a core-shell structure including a core comprising the at least one oxygen sensitive compound in a first polymer, and a shell comprising a second polymer.

12. The device of claim 1, wherein the detector is a photodiode or a spectrometer.

13. The device of claim 1, wherein the detector comprises a preamplifier configured to amplify a signal based on the radiation emitted from the nanofiber.

14. The device of claim 1, wherein the enclosure is made of an opaque material, and the opening is a hole or a slot located above or below the sensing element.

15. The device of claim 1, wherein the enclosure is in tubular shape, and the light source and the detector are disposed on two opposite ends of the enclosure.

16. An apparatus comprising:
an enclosure defining an opening for introducing a sample for testing;
a light source configured to provide light;
a sensing element comprising nanofiber and disposed inside the enclosure, the nanofiber comprising at least one oxygen sensitive compound and configured to receive the light from the light source and then emit radiation;
a detector configured to detect the radiation emitted from the nanofiber and provide a measured concentration of oxygen in the sample; and
at least one heating element for locally increasing temperature comprising an electrically conductive and optically transparent coating, and disposed in close proximity to or in contact with the sensing element.

17. The apparatus of claim 16, wherein the electrically conductive and optically transparent coating comprises indium tin oxide.

18. The apparatus of claim 16, further comprising:
a porous mask comprising expanded polytetrafluoroethylene (ePTFE), disposed on a surface of the enclosure and covering the opening.

19. The apparatus of claim 16, further comprising:
at least one optical filter disposed between the sensing element and the light source or the detector.

20. The apparatus of claim 16, further comprising:
a light pipe as a pathway for the light from the light source or the radiation emitted from the nanofiber

21. The apparatus of claim 16, wherein the apparatus is a mouth guard sized and shaped to be wearable by a human subject.

22. A method of using the device of claim 1, comprising:
introducing a sample being a gas or a liquid adjacent to the opening of the enclosure so as to contact the sample with the sensing element;
exciting the nanofiber using the light from the light source continuously or discontinuously; and
detecting the radiation emitted from the nanofiber so as to provide a measured concentration of oxygen in the sample.

23. The method of claim 22, wherein the nanofiber is excited discontinuously in a pulsed mode.

24. The method of claim 22, further comprising heating at least one heating element disposed in close proximity to or in contact with the sensing element to prevent moisture condensation, the at least one heating element comprising an electrically conductive and optically transparent coating.
